(19) United States
(12) Patent Application Publication
(10) Pub. No.: US 2013/0220234 A1
(43) Pub. Date: Aug. 29, 2013

(54) FLOORLESS SHELTER

(71) Applicant: Karen Louise Stevens, Normal, IL (US)

(72) Inventor: Karen Louise Stevens, Normal, IL (US)

(21) Appl. No.: 13/673,876

(22) Filed: Nov. 9, 2012

Related U.S. Application Data
(60) Provisional application No. 61/603,144, filed on Feb. 24, 2012.

(51) Int. Cl. A01K 15/02; E04H 15/14; A01K 67/00; E04H 15/56

(52) U.S. Cl. CPC A01K 15/02; E04H 15/56; E04H 15/14; A01K 67/00
USPC 119/712; 135/116; 135/93

(57) ABSTRACT

A floorless shelter has a closable door and a rear ventilation window that offers shelter in inclement weather for an owner/trainer and canine during housebreaking, as well as for use on a continuing basis. The floorless shelter also provides a specific area, recommended for housebreaking, which reduces distractions because it is enclosed. After repeatedly being taken to the structure, especially in inclement weather, the canine becomes conditioned to use the specific area and appreciate the protection from the elements that the structure provides. The floorless shelter makes the housebreaking and continued use both convenient and successful.

### CROSS-REFERENCE TO RELATED APPLICATION

This application claims the benefit of priority of U.S. provisional application No. 61/603,144, filed Feb. 24, 2012, the contents of which are herein incorporated by reference.

### BACKGROUND OF THE INVENTION

The present invention relates to shelters and, more particularly, to a floorless tent-like shelter, especially for use in inclement weather conditions and used primarily for housebreaking canines outside.

Housebreaking canines is often done by establishing a specific area outside and limiting distractions. Many canines, however, are reluctant to go outside to potty in inclement weather. This includes canines that are already housebroken as well as those canines that are still being “potty trained”.

As can be seen, there is a need for a shelter for canines that can be used for providing a designated, distraction-free area for housebreaking canines outside and, for already housebroken canines, providing shelter for canines reluctant to venture outside in inclement weather.

### SUMMARY OF THE INVENTION

In one aspect of the present invention, a floorless shelter comprises a main body defining an interior of the floorless shelter; an opening in the main body; a door openable and closable over the opening; a floor stabilizer disposed about and attached to an outer periphery of the main body; and a plurality of grommets defining holes through the floor stabilizer.

In another aspect of the present invention, a floorless shelter comprises a main body defining an interior of the floorless shelter; an opening in the main body; a door openable and closable over the opening; a zipper disposed to permit opening and closing of the door; a floor stabilizer disposed about and attached to an outer periphery of the main body; a plurality of grommets defining holes through the floor stabilizer; and a ventilation window disposed in the main body.

In a further aspect of the present invention, a method for housebreaking a canine comprises leading the canine into a floorless shelter, the floorless shelter having a main body defining an interior of the floorless shelter, an opening in the main body, a door openable and closable over the opening, a floor stabilizer disposed about and attached to an outer periphery of the main body, and a plurality of grommets defining holes through the floor stabilizer; closing the door over the opening to prevent the canine from exiting the floorless shelter prematurely; giving a command to the canine to go potty; and leading the canine out of the floorless shelter when the canine is finished.

These and other features, aspects and advantages of the present invention will become better understood with reference to the following drawings, description and claims.

### BRIEF DESCRIPTION OF THE DRAWINGS

FIG. 1 is a front perspective view of a floorless shelter according to an exemplary embodiment of the present invention;

FIG. 2 is aside view of the floorless shelter of FIG. 1;

FIG. 3 is a front view of the floorless shelter of FIG. 1;

FIG. 4 is a side perspective view of the floorless shelter of FIG. 1;

FIG. 5 is a left side view of the floorless shelter of FIG. 1;

FIG. 6 is a right side view of the floorless shelter of FIG. 1;

FIG. 7 is a top view of the floorless shelter of FIG. 1;

FIG. 8 is a rear view of the floorless shelter of FIG. 1;

FIG. 9 is a rear perspective view of the floorless shelter of FIG. 1; and

FIG. 10 is a bottom view of the floorless shelter of FIG. 1.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention provides a floorless shelter having a closable door and a rear ventilation window that offers shelter in inclement weather for an owner/trainer and canine during housebreaking, as well as for use on a continuing basis. The floorless shelter also provides a specific area, recommended for housebreaking, which reduces distractions because it is enclosed. After repeatedly being taken to the structure, especially in inclement weather, the canine becomes conditioned to use the specific area and appreciate the protection from the elements that the structure provides. The floorless shelter makes the housebreaking and continued use both convenient and successful.

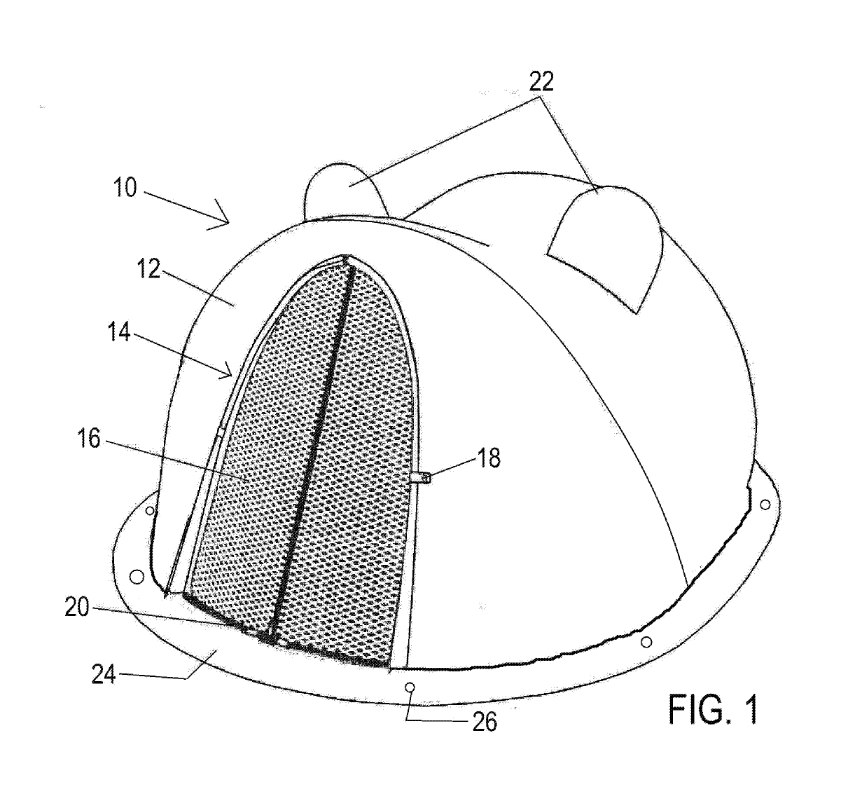

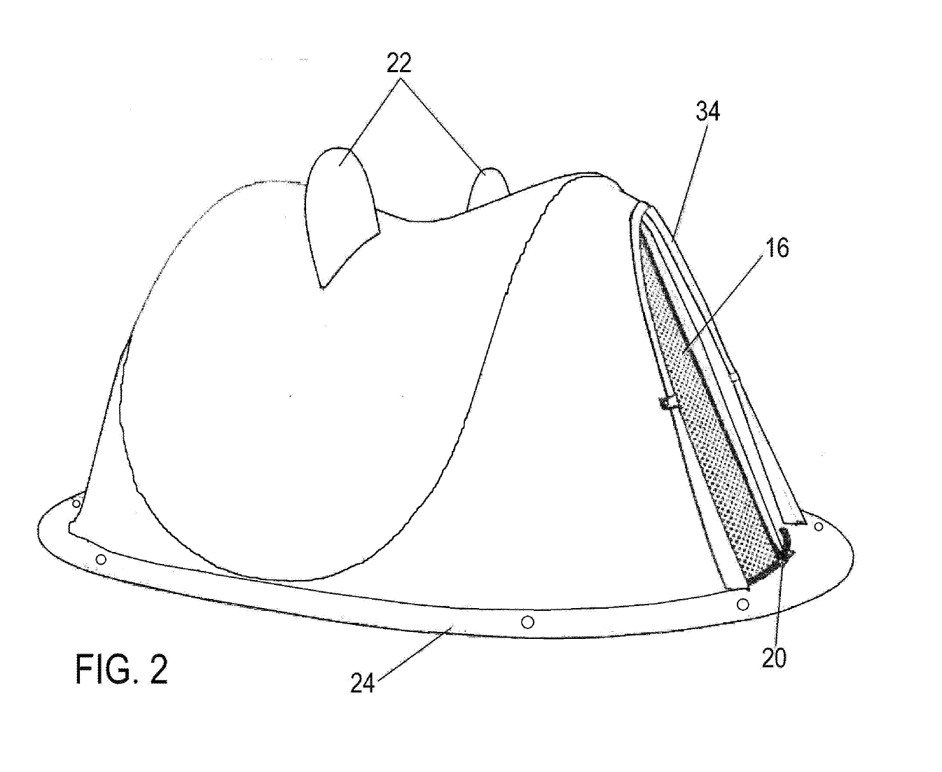

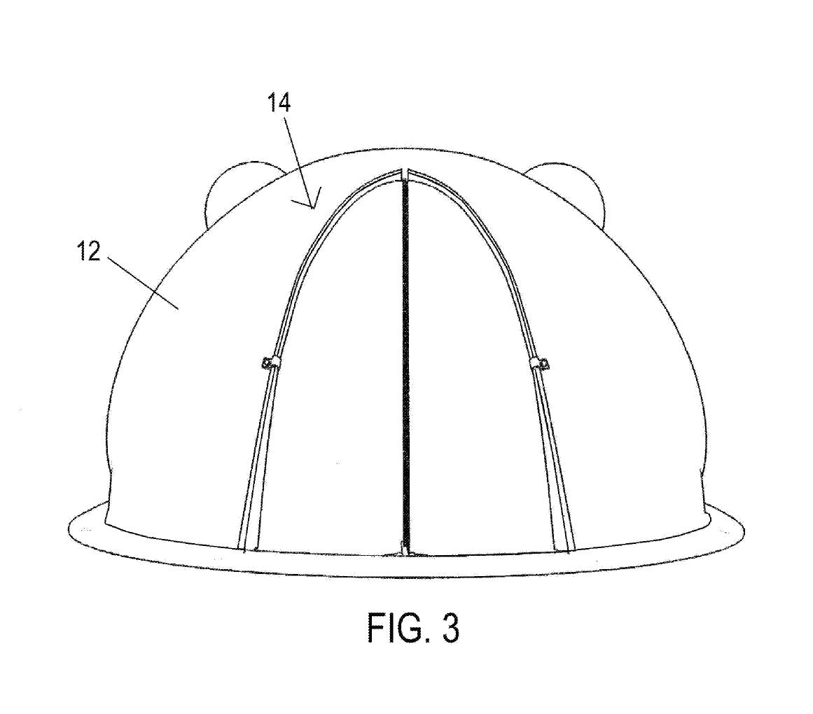

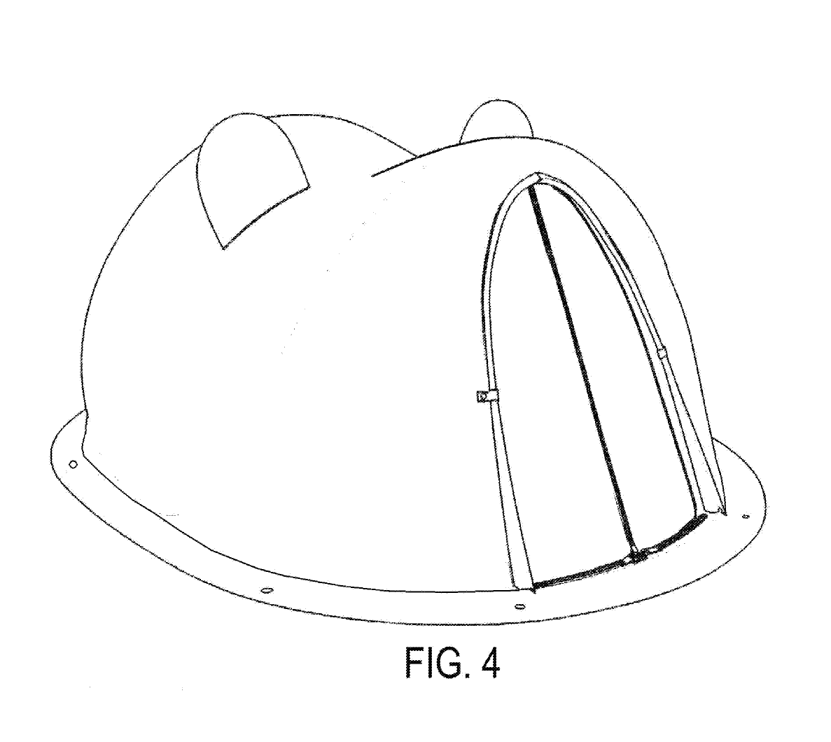

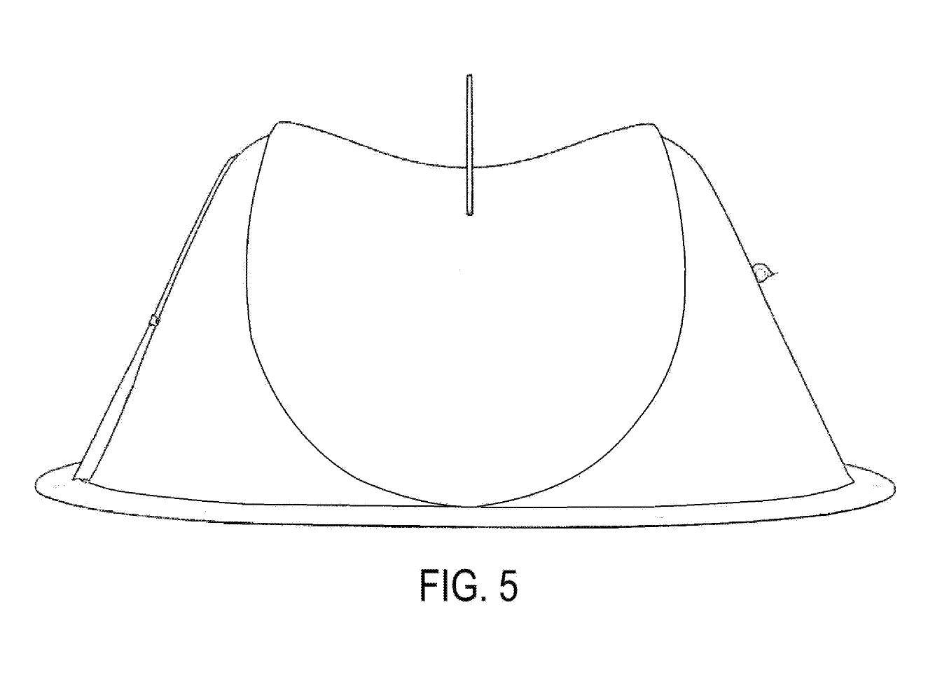

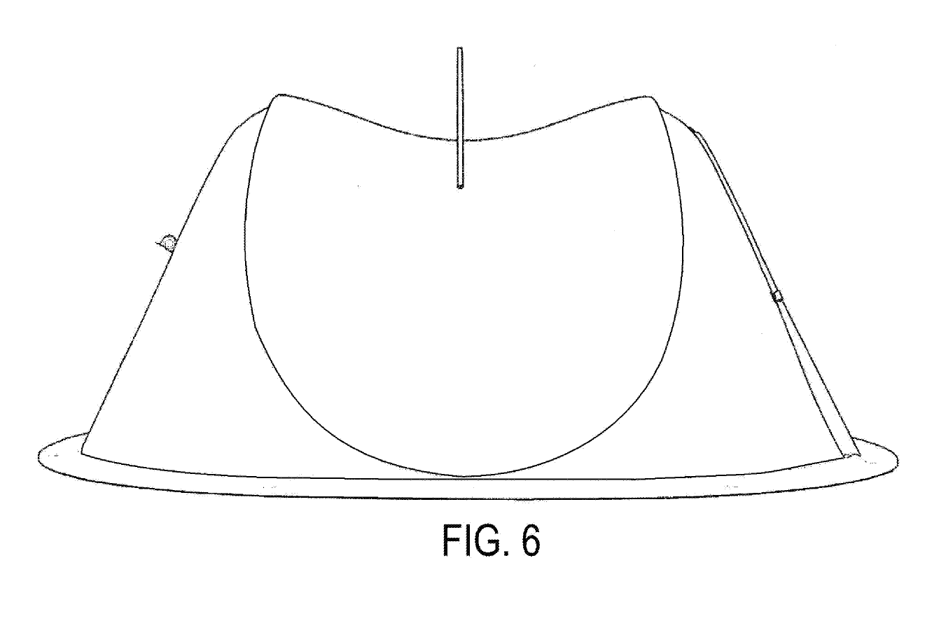

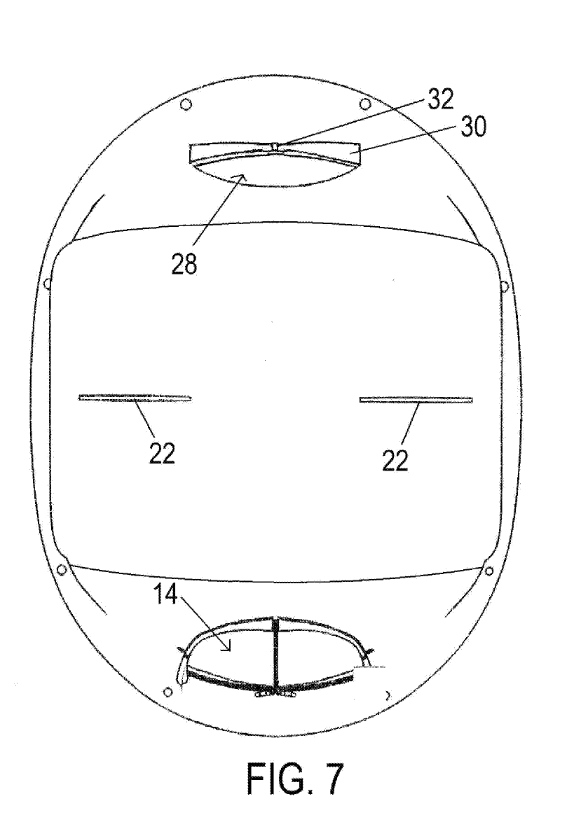

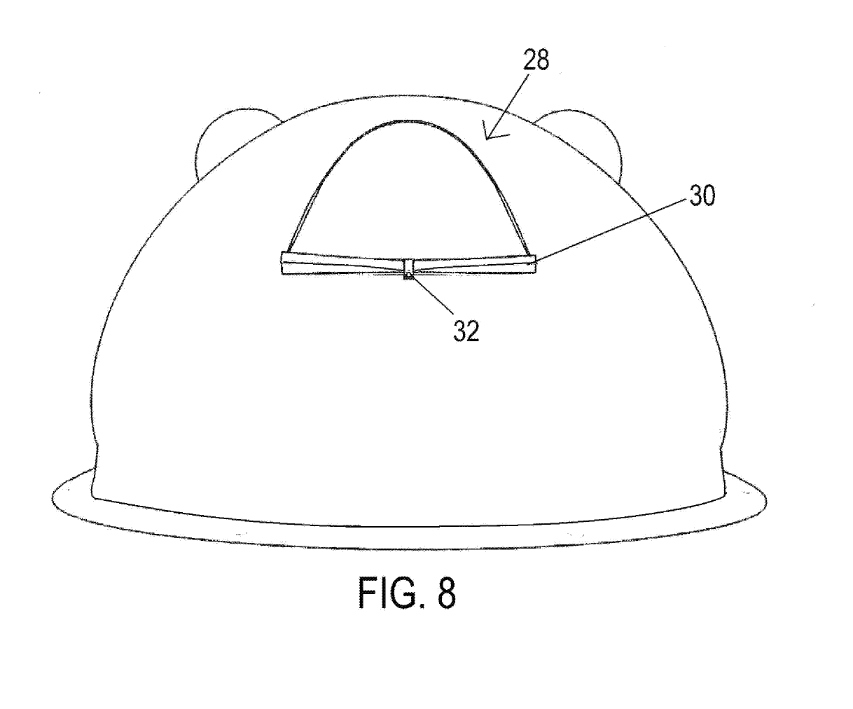

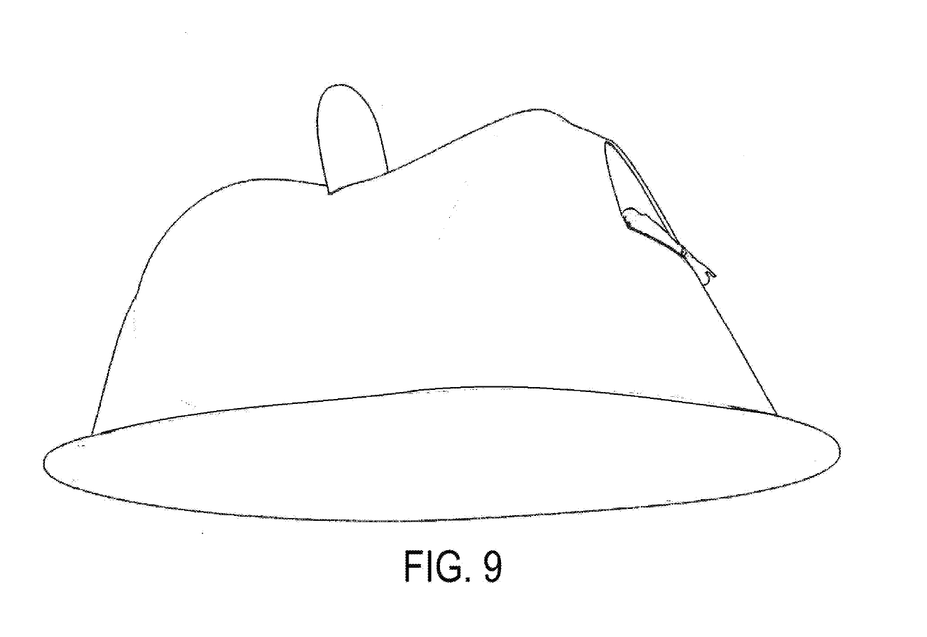

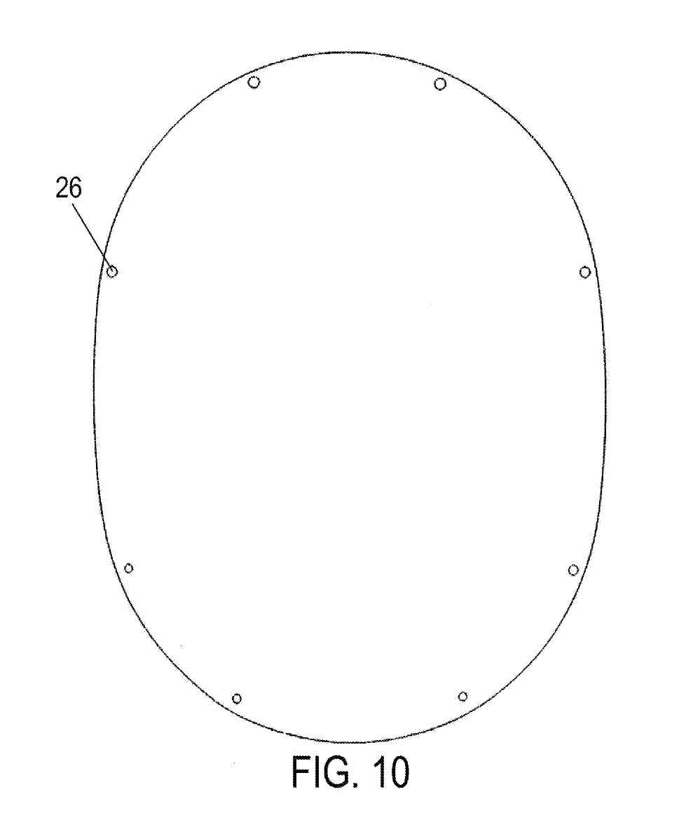

Referring now to FIGS. 1 through 10, a floorless shelter 10 can include a main body 12 having an opening 14 on a front end thereof. The opening 14 can have a door 16 removably disposed to cover the opening 14. The door 16 can be, for example, a piece of material, a netting material, a mesh material, or the like. The door 16 can open and close to allow a canine and/or an owner/trainer to enter and exit the shelter 10. Fasteners 18 can be used to hold sides of the door 16 and an opening/closing mechanism, such as a zipper 20, can be disposed to open and close the door 16 over the opening 14. A door cover 34 can be held by the fasteners 18 and can cover the opening and the door 16, if desired.

The main body 12 can be made in various colors, shapes and sizes. Ears 22 can be disposed along the top of the main body 12. The ears 22 can resemble various dog ears, for example. The main body 12 can be made in various colors and patterns, such as animal prints, dog breed colors, and the like. The main body 12 can be made of a material that would not allow water to penetrate, such as a conventional tent material, for example.

The main body 12 can be made in various sizes and shapes, such as oval, round, rectangular, square, or other geometric or non-geometric shapes. The inside floor area under the main body can be from about 20 to about 100 square feet. For example, the inside floor area could be defined by a rounded square shape about 7 feet by 7 feet in size. The height of the main body 12 can also vary. Typically, the height can be large enough to allow the owner/trainer to easily enter and exit, however, shorter heights, designed only for the canine, are contemplated within the scope of the present invention. Typically, the height of the main body can be from about 4 feet to about 7 feet.

A floor stabilizer 24 can be disposed about an outer periphery of the main body 12. The floor stabilizer 24 can be a study material to provide for base stabilization. The floor stabilizer 24 can be connected to or formed integrally with the main body 12. Grommets 26 can be disposed about the floor stabilizer 24 to provide a method for staking down the shelter 10 to prevent movement thereof.

A rear side of the main body 12 can include an opening 28 for ventilation. The opening 28 can be covered with, for example, a mesh material that can deter rain or snow from entering the shelter, but can allow air flow therethrough. The opening can include a closure 30 that can roll down and be secured open by a strap 32. If desired, the opening 28 can be closed with the closure 30 by, for example, zipping the closure 30 over the opening 28.

To use the floorless shelter 10, it can be set up like a pop-tent and staked down to the ground through the grommets 26 through the floor stabilizer 24. Once in place, a user can lead or carry their canine into the structure and, at least during the initial stages of training, zip closed the door while inside. The user can give the “go potty” command and, after the canine goes, can praise them and provide a treat as a reward. As these steps are consistently repeated with a pup and/or an adult canine, the canine will establish this area as their place to go. After the canine is housebroken, the shelter can be used to provide shelter to canines to potty outside, even in inclement weather. A large majority of canines of all ages and sizes are reluctant, especially in inclement weather, to be trained to be housebroken or even go potty outside, even when they are housebroken already, in anything other than good weather conditions.

The floorless shelter of the present invention can include various options within the scope of the present invention. For example, the floorless shelter can include one or more pockets inside the shelter, typically disposed on the inside of the main body. The pockets can be used, for example, to hold canine treats to be provided as a reward to the canine.

It should be understood, of course, that the foregoing relates to exemplary embodiments of the invention and that modifications may be made without departing from the spirit and scope of the invention as set forth in the following claims.

What is claimed is:

1. A floorless shelter comprising:
a main body defining an interior of the floorless shelter;
an opening in the main body;
a door openable and closable over the opening;
a floor stabilizer disposed about and attached to an outer periphery of the main body; and
a plurality of grommets defining holes through the floor stabilizer.

2. The floorless shelter of claim 1, further comprising fasteners disposed about the opening and securing the door thereto.

3. The floorless shelter of claim 1, further comprising an opening/closing mechanism operable to open and close the door.

4. The floorless shelter of claim 3, wherein the opening/closing mechanism includes a zipper.

5. The floorless shelter of claim 1, further comprising a ventilation window disposed in the main body.

6. The floorless shelter of claim 5, wherein the ventilation window is disposed opposite the door.

7. The floorless shelter of claim 5, further comprising a cover operable to cover the ventilation window.

8. The floorless shelter of claim 1, wherein the door is formed of a mesh material.

9. A floorless shelter comprising:
a main body defining an interior of the floorless shelter;
an opening in the main body;
a door openable and closable over the opening;
a zipper disposed to permit opening and closing of the door;
a floor stabilizer disposed about and attached to an outer periphery of the main body;
a plurality of grommets defining holes through the floor stabilizer; and
a ventilation window disposed in the main body.

10. The floorless shelter of claim 9, further comprising fasteners disposed about the opening and securing the door thereto.

11. The floorless shelter of claim 9, further comprising a cover operable to cover the ventilation window.

12. The floorless shelter of claim 9, wherein the door is formed of a mesh material.

13. A method for housebreaking a canine, comprising:
leading the canine into a floorless shelter, the floorless shelter having a main body defining an interior of the floorless shelter, an opening in the main body, a door openable and closable over the opening, a floor stabilizer disposed about and attached to an outer periphery of the main body, and a plurality of grommets defining holes through the floor stabilizer;
closing the door over the opening to prevent the canine from exiting the floorless shelter prematurely;
giving a command to the canine to go potty; and
leading the canine out of the floorless shelter when the canine is finished.

14. The method of claim 13, further comprising providing a treat to the canine when the canine is finished.
